(19) United States
(12) Patent Application Publication
(10) Pub. No.: US 2013/0172659 A1
(43) Pub. Date: Jul. 4, 2013

(54) Electromagnetic Deep Tissue Excitation

(71) Applicant: Oron Zachar, Tel Aviv (IL)

(72) Inventor: Oron Zachar, Tel Aviv (IL)

(21) Appl. No.: 13/341,962

(22) Filed: Dec. 31, 2011

(51) Int. Cl. A61N 2/02

(52) U.S. Cl. 600/13

(57) ABSTRACT

The present invention delineates methods and devices for non-invasive generation of concentrated electric fields within a subject animal body using electromagnetic field sources placed outside of said animal body. Said sources, placed externally of the body, are operable to induce electric currents within the body of that subject and particularly in the brain. In particular embodiments, devices according to the invention can be used for focused deep brain stimulation (DBS) of selected brain regions with minimal effect on undesired other brain regions.

### FIELD OF THE INVENTION

The present invention relates to methods and devices for non-invasive brain stimulation using electromagnetic field sources. Said sources, placed externally of the body, are operable to induce electric currents within the body of that subject and particularly in the brain.

### BACKGROUND OF THE INVENTION

Electromagnets are capable of inducing electric fields in most biological tissues. Biological cells normally maintain a potential, Vr, in which the interior of the cell is negative with respect to its exterior. Typical values of Vr for nerve and muscle cells are −65 and −90 mV respectively. Considering the membrane potential (≈0.1 V) and thickness (≈10⁻⁸ m), the electric field developed across the resting membrane is around 10⁷ V/m. The conductivity of the excitable membrane is controlled by this enormous electric field. Disturbances from the resting condition can lead to profound changes in the membrane's electrical properties, and ultimately initiate the functional responses of nerve and muscle.

Particular value and benefit has been proposed to arise from electromagnetic stimulation of brain tissue. Representative examples of the present art of electromagnetic brain stimulation is provided within patents and applications, and references therein of—

| | |
|---|---|
| U.S. 2009/0287108 patent | SYSTEM FOR STUDY AND |
| application | TREATMENT OF BEHAVIOR |
| | DYSREGULATION |
| U.S. Pat. No. 7,577,481 | Methods and apparatus for effectuating |
| granted patent | a lasting change in a neural-function of |
| | a patient |
| U.S. 2009/0156884 patent | TRANSCRANIAL MAGNET |
| application | STIMULATION OF DEEP BRAIN |
| | TARGETS |
| U.S. 2009/0105521 patent | Systems and methods for assessing |
| application | and treating medical conditions related |
| | to the central nervous system and for |
| | enhancing cognitive functions |
| U.S. 2009/0105784 patent | MINIATURE WIRELESS SYSTEM |
| application | FOR DEEP BRAIN STIMULATION |
| WO 2007/007058 patent | METHOD AND APPARATUS FOR |
| application | REGULATING BLOOD PRESSURE |

Highly localized electromagnetic stimulation of finely targeted brain regions is possible with implanted electrodes inside the brain. Yet, this requires invasive surgical procedures, which are costly and risky, for each desired brain location for stimulation.

Therefore, there is a need for non-invasive brain stimulation methods. Using external field sources, transcranial magnetic stimulation differs from transcranial electric stimulation because during magnetic stimulation the electric field is parallel to the brain surface, whereas during electric stimulation the electric field has components both parallel and perpendicular to the brain surface.

The prominent present art method for non-invasive brain stimulation is by using magnetic coils (e.g., “FIG. 8 coils”), and is commonly referred to as “magnetic brain stimulation”. Yet, these produce a peak of magnetic fields only a short distance of about 2 cm from the sculpt surface. Hence, deep regions of the brain cannot be stimulated with present art technology.

The need for non-invasive deep brain stimulation (DBS) remains. Present art so-called DBS, with more complicated coil structures as described in US application 20080312706, actually reach only a peak only about 1 cm deeper than common FIG. 8 coils. Moreover, the coil peak is shallow with a peak height no more than 2× the minimum field between the coil center and the peak. Further deficit of such coils is that the peak is very broad, with half-width of several centimeters, thereby wide regions of the brain are stimulated.

Altogether, focused small region deep brain stimulation is not achieved by any present art non-invasive brain stimulation method. The present invention is providing exactly such a method and apparatus.

Remarkably, it has been thought to be proven mathematically that “at the frequencies generally proposed for extracranial stimulation of the brain, it is not possible, using any superposition of external current sources, to produce a three-dimensional local maximum of the electric field strength inside the brain” (Heller & van-Hulsteyn, Biophys. Journal, Vol. 63 1992 p. 129-138). In contrast, with the present invention we show that the analysis of the above noted article has a more limited validity than previously thought—we do present a method of producing highly focused and highly peaked deep brain stimulation electromagnetic fields.

The bulk of prior art non-invasive brain stimulation relies on the method of using various forms of coils to produce an initial static magnetic field using an initial steady current in the coils. So long as the magnetic field remains constant there is no brain stimulation. The brain stimulation is induced when typically the current in the coils is sharply interrupted, leading to sudden drop of the magnetic field intensity. The change in magnetic fields within the brain induces local currents. The intensity of induced currents is proportional to the initial local amplitude of the static magnetic fields.

Another type of treatment is transcranial magnetic stimulation (TMS), which involves producing a high-powered magnetic field adjacent to the exterior of the scalp over an area of the cortex. Although TMS appears to be able to produce a change in the underlying cortex beyond the time of actual stimulation, TMS is not presently effective for treating many patients because the existing delivery systems are not practical for applying stimulation over an adequate period of time. TMS systems, for example, are relatively complex and require stimulation treatments to be performed by a healthcare professional in a hospital or physician's office. TMS systems also may not be reliable for longer-term therapies because it is difficult to (a) accurately localize the region of stimulation in a reproducible manner, and (b) hold the device in the correct position over the cranium for a long period, especially when a patient moves or during rehabilitation. Furthermore, current TMS systems generally do not sufficiently focus the electromagnetic energy on the desired region of the cortex for many applications. As such, the potential therapeutic benefit of TMS using existing equipment is relatively limited.

Other methods of brain stimulation include transcranial electrical stimulation (TES), which involves placing an electrode on the exterior of the scalp and delivering an electrical current to the brain through the scalp and skull. Patents directed to TES include: U.S. Pat. No. 5,540,736 issued to Haimovich et al. (for providing analgesia); U.S. Pat. No. 4,140,133 issued to Katrubin et al. (for providing anesthesia); U.S. Pat. No. 4,646,744 issued to Capel (for treating drug addiction, appetite disorders, stress, insomnia and pain); and U.S. Pat. No. 4,844,075 issued to Liss et al. (for treating pain and motor dysfunction associated with cerebral palsy). TES, however, is not widely used because the patients experience a great amount of pain and the electrical field is difficult to direct or focus accurately.

Direct and indirect electrical stimulation of the central nervous system has also been proposed to treat a variety of disorders and conditions. For example, U.S. Pat. No. 5,938,688 issued to Schiff notes that the phenomenon of neuroplasticity may be harnessed and enhanced to treat cognitive disorders related to brain injuries caused by trauma or stroke. Schiffs implant is designed to increase the level of arousal of a comatose patient by stimulating deep brain centers involved in consciousness. To do this, Schiffs invention involves electrically stimulating at least a portion of the patient's intralaminar nuclei (i.e., the deep brain) using, e.g., an implantable multipolar electrode and either an implantable pulse generator or an external radiofrequency controlled pulse generator. Schiffs deep brain implant is highly invasive, however, and could involve serious complications for the patient.

A recent good review of the prior art of brain stimulation can be found in US patent application 2006/0200206. Also within it describe is a method of brain stimulation comprising of an electrodes implanting procedure; involving positioning first and second electrodes at the identified stimulation site, and a stimulating procedure involving applying an electrical current between the first and second electrodes.

The present invention also relates to methods of inducing, stimulating, or enhancing particular brainwaves states. Present art of such brainwaves control is mostly based on biofeedback methods. The U.S. Pat. No. 5,406,957 and patent application WO 1999/042032 exemplify the present state of the art of brainwaves stimulation and control. Conventional EEG biofeedback methods and apparatus have referenced brainwave activity in terms of large bands of EEG. As such, brainwave activity has traditionally been classified as follows: delta waves lie in the frequency range of 0 to 3.5 Hz; theta waves lie in the frequency range of 4 to 7 Hz; alpha waves lie in the frequency range of 8 to 13 Hz; beta waves lie in the frequency range above 13 HHz; and sensorimotor rhythm (SMR) waves lie in the frequency range of 12 to 15 Hz. More refined subdivision of brainwaves band is well known in the scientific literature. Several patents have been directed to monitoring EEG in terms of the sensed amplitudes and percentages of alpha, theta, beta, delta and SMR waves.

Biofeedback methods are prominently reliant upon active attention and intentional concentration of the human subject. As such prior art of brainwaves stimulation is sensitive to irregular attention of both within individual subjects and differences between human subjects. Moreover, animal tests are nearly impossible with such intentional biofeedback methods. There is a need and preference for more controlled, quantitative, and objective methods of stimulating particular brainwaves without relying on biofeedback will and participation of the animal subject.

Pain Relief Neuro-Stimulation.

Neurostimulation methods used today for pain relief have inherent drawbacks. The commonly used methods are based on the delivery of stimulation to wide areas around the target, forcing clinicians to use low level stimulation that do not efficiently activate the endorphin mechanism and compromises the quality of the treatment. Other methods are based on manually identifying and stimulating the targets. These deliver a more pinpoint treatment for activating the endorphin mechanism, but are nonetheless less efficient as they are performed manually and are heavily dependent on the operator's level of skill and prior knowledge. Activating the endorphin mechanism using electrotherapy requires the provision of strong, low-rate electrical pulses (SLR-TENS or ALTENS) at appropriate locations for producing analgesia by stimulating of the A δ nerve afferent neurons (III fibers). This will induce the release of the body's own pain killers: natural endorphins, cortisol and serotonin hormone. Effective stimulation requires targeting the location of the nerve. Preferably, an Automated Pinpoint Treatment would require both the technology capable of auto localizing the targets, and a precise method of treating the targets alone.

The present invention also relates to radio-frequency ablation surgery. The term “radiofrequency ablation probe” refers to a class of medical devices operating between 460-550 kHz that deliver therapeutic energy into soft tissues. The intent of these devices is to thermally necrose tissue by raising targeted tissue temperatures to approximately 100° C. for a period of 10-15 minutes [1,2]. Ablation probes are inserted percutaneously or subdermally into tissues where cancerous tumors have been identified. Once the probes are positioned, radiofrequency energy is delivered through the probe, into surrounding tissue, and to an electrical ground pad that is applied to the skin of the patient. Electrosurgical devices and radiofrequency ablation probes both operate within the same frequency range and transfer energy in similar ways, but differ in the shape of stimulation waveforms. Radiofrequency ablation devices utilize a continuous sinusoidal waveform. (Representative discussion can be found in the article “Finite Element Analysis of Hepatic Radiofrequency Ablation Probes using Temperature-Dependent Electrical Conductivity” by Isaac Chang, BioMedical Engineering OnLine 2003, 2:12). Prior art of radiofrequency ablation is exemplified in US patent applications 20020058935, 20080103497.

The invention also relates to method of inducing local hyperthermia of cancer tissue. The intent of microwave hyperthermia devices (commonly operating at 27 MHz or 2.45 GHz) is to raise the temperature of timorous tissues to between 43-45° C. for extended periods of time on the order of hours. Microwave hyperthermia devices deliver energy at low doses of energy over long periods of time; whereas, radiofrequency ablation devices deliver high doses of energy for short periods of time. Prior art of radiofrequency hyperthermia induction is exemplified in U.S. Pat. Nos. 4,674,481, 5,284,144, 6,016,452, 6,652,519. In present art, inducing hyperthermia with electromagnetic radiation to restricted deep tissue areas is possible only with invasive needles or catheters. Non-invasive methods, such as described in U.S. Pat. No. 4,674,481, cannot provide good focus to deep tissue.

An attempt to produce a hyperthermia apparatus having three-dimensional focusing of electromagnetic radiation is provided in U.S. Pat. No. 5,097,844. Yet the focusing is not effective enough with this method. The normal operating frequency range for this system for the torso portion of an adult is between about 50 to 1000 MHz. It is most useful between 60 to 220 MHz where the penetration characteristics of the body are deeper.

The invention also relates to methods of overcoming the blood-brain barrier using electromagnetic fields. Prior art of this subject matter is exemplified in U.S. Pat. No. 7,120,489 and references therein. In prior art electromagnetic stimulation of selected nerves is achieved by invasive electrodes.

The invention also relates to methods of increase cerebral blood flow (CBF) of the subject, so as to treat a condition of the subject. Prior art of this subject matter is exemplified in US patent application 2004/0220644 and references therein. In prior art electromagnetic stimulation of selected nerves is achieved by invasive electrodes.

It has been known for many years that improved healing rates can be achieved by applying r.f. electromagnetic fields to wounded tissue. The therapeutic effects were considered to be due to heating of the tissue by the field and prior therapy apparatus has been configured to produce r.f. energy levels for tissue heating either on the surface or deep into the tissue. This heating technique is known as diathermy. It is known to pulse the r.f. field produced by diathermy apparatus. A specific example of the healing effects achieved with a pulsed field diathermy apparatus is given in “A Trial Involving the Use of Pulsed Electromagnetic Therapy on Children Undergoing Orchidopexy” R. H. C. Bentall and H. B. Eckstein, Zeitschrift fur Kinderchirurgie and Grenzgebiete p. 380-398 November 1975. Heretofore the pulsed electromagetic field has been produced by hospital or laboratory based equipment comprising an electrical signal generator which feeds an induction coil mounted on a stand positioned adjacent an area of a patient to be treated.

The invention further relates to radiofrequency stimulation of blood coagulation. The prior art on this subject matter is exemplified in patent application WO 2004/107994.

The present invention makes use of an elliptical mirror element. Discussion of elliptical mirrors in prior art can be found in—

| | |
|---|---|
| System of mirrors for guiding an | U.S. Pat. No. 5,042,931 |
| electromagnetic wave | granted patent |
| Power generation devices and | US 2006/0046907 |
| methods | patent application |

The invention relates to propagation of electromagnetic waves in biological tissue. Hence it is relevant to review the scientific knowledge on that topic.

Tissue is composed primarily of water. The human body is between 50% and 70% water by weight. FIG. 4 shows the dielectric constant ∈ dispersion of biological tissue and compares it to pure water [This information comes from Kenneth R. Foster and Herman P. Schwan, “Dielectric properties of tissues and biological materials: a critical review” Crit. Rev. in Biomed. Engr., 17, pp. 25-104, 1989]. At audio frequencies, the alpha dispersion is dominated by counterion polarization effects. At HF frequencies, the beta dispersion is dominated by interfacial polarization of the cell walls. At radio and microwave frequencies above about 100 MHz, the dispersion characteristics of water and tissue are well matched. This is the so called gamma dispersion where the Debye relaxation of water molecules dominates the dispersion of tissue.

The dielectric constant (which is often dependent on wavelength) is simply the square of the (complex) refractive index in a non-magnetic medium (one with a relative permeability of unity). The refractive index is used for optics in Fresnel equations and Snell's law; while the dielectric constant is used in Maxwell's equations and electronics. Where \tilde{ε}, ε₁, ε₂, n, and κ are functions of wavelength:

\tilde{ε}=ε₁+iε₂=(n+iκ)². (Eq. 1)

Conversion between refractive index “n” and dielectric constant ε is done by:

$$\begin{matrix}{ε}_{1} = {n}^{2} - {κ}^{2} & ( Eq . 2 ) \\ {ε}_{2} = 2 n κ & ( Eq . 3 ) \\ n = \sqrt{\frac{\sqrt{{ε}_{1}^{2} + {ε}_{2}^{2}} + {ε}_{1}}{2}} & ( Eq . 4 ) \\ κ = \sqrt{\frac{\sqrt{{ε}_{1}^{2} + {ε}_{2}^{2}} - {ε}_{1}}{2}} . & ( Eq . 5 )\end{matrix}$$

In connection with FIG. 4, we note that the index of refraction is approximately equal to the real part of the dielectric constant, i.e., n˜Re[ε], so the verticle axis of FIG. 4 can be regarded also as designating n².

The known Snell's principle of refraction is that light incoming from medium n1 at an angle θ₁ to the normal of the interface to medium n2 is continuing in medium n2 at angle θ₂ according to

$$\frac{sin {θ}_{1}}{sin {θ}_{2}} = \frac{{v}_{1}}{{v}_{2}} = \frac{{n}_{2}}{{n}_{1}} \qquad ( Eq . 6 )$$

Altogether, there is a need for more targeted and focused non-invasive deep tissue electromagnetic stimulation methods, with minimum effect to undesired regions.

### SUMMARY OF THE INVENTION

The present invention delineates methods and devices for non-invasive generation of concentrated electric fields within a subject animal body using electromagnetic field sources placed outside of said animal body. Said sources, placed externally of the body, are operable to induce electric currents within the body of that subject and particularly in the brain.

The invention method applies a generation of contracting sections of squai spherical wave fronts. In preferred embodiments, further introducing an index of refraction mediating medium between the human subject body and the radiation source.

Prior art of generating concentrating spherical waves was implemented for applications in which the radiation propagated in uniform free space. In contrast, the present invention aims to produce quasi-spherical concentrating waves in human tissue medium “n2” while the radiation sources are located in different medium “n1” (e.g., air) outside of said tissue. For such a mode of application, we identify a key problem that spherical waves get deformed and defocused in transition from one medium to another, particularly from free space to human tissue (see FIG. 6b). The present invention introduces a method and apparatus for minimizing said deformation and defocusing, and thereby enhancing the focus and improving the efficacy of tissue stimulation by method of concentrating spherical wave electromagnetic radiation.

In preferred embodiments, as illustrated in FIG. 2, the invention method of is based on generating a concentrating section of quasi-spherical wave front by: introducing a relatively small source 135 emitting electromagnetic waves (e.g., an emission antenna, optical fiber, etc. . . . ) outside of a subject body 190; and placing said source at one of the geometric focal points (F1) 121 of a section of an elliptical mirror 110, reflecting at least a portion of the emitted radiation from said elliptical mirror 110; placing the a body part 190 of a human subject in a location such that a desired pre-selected body region of the subject is located at the second geometrical focal point (F2) 122 of said elliptical mirror 110; thereby creating a focused image of the source 135 radiation at the region of said F2 focal point within the dody of said subject animal. The smaller is the radiation source 135 the closer to ideal spherical is the produced wave.

As illustrated in FIG. 2, it is known in the general prior art of elliptical mirrors, an ideal point source 135 at one focal point 121 (F1) produces a spherical wave front which concentrate towards the second focal point 122 (F2) of the elliptical reflecting surface. Yet, as illustrated in FIG. 6b, a new problem arises when the source is placed in one medium with index of refraction “n1”, while the second geometrical focal point F2 of the ellipse is located within a medium with a different index of refraction “n2”—the incident spherical wave from medium n1 is deformed and does not converge to the focal point F2 in medium n2.

In particular, for putative non-invasive medical applications where the electromagnetic radiation source 121 is placed in air medium with index of refraction n1˜1, and the target human organ is placed within a human body of average index n2>n1, then the radiation emitted from any prior art elliptical reflector will be defocused and will not get concentrated in the second geometrical focal point F2 of the elliptical reflecting surface 110. As illustrated in FIG. 6b, a spherical wave front 610 is incident on the boundary surface between medium n1 (air) and medium n2 (body tissue). A part 601 of the wave front arriving at an angle α1 is refracted upon entering medium n2, and continue along a path 602 at a different angle α2 and thereby arriving to location 635 which is distant from the intended location 122 of the focal point F2. Each part of the spherical wave front 610 is incident at a different angle on the boundary surface, and gets refracted at a different angle. Consequently, the radiation is smeared over a wide region 630 in the medium n2 instead of being concentrated around F2. The present invention introduces a method and apparatus to overcome this problem.

The invention introduces a method and apparatus which reduce the above noted deformation of the incident spherical wave front 610 upon entering a human tissue medium, and enhances its concentration towards the geometrical focal point F2 (inside a human tissue medium) from the reflecting elliptical surface 110 positioned outside the human body medium n2. The fundamental physical principle of the invention method is schematically represented in FIG. 6c.

As illustrated in FIG. 6c, according to the present invention, an index matching secondary medium 565 with refraction index n3 is confined on at least one side by a thin transparent spherical section shell 560 and filling tightly down to the boundary surface with the target medium n2. The spherical shell 560 is constructed and positioned such that its geometrical center coincides with the focal point F2 of the elliptical reflecting surface 110.

In the ideal case, the secondary medium 565 refraction index is perfectly matching the n2, i.e., n3=n2. Then the incident spherical wave front 610 is not deformed entering human tissue medium n2 and continues to get concentrated towards the desired focal point F2, as illustrated in FIG. 6c.

In less ideal cases the index matching secondary medium 565 is not perfectly matching to the human body average n2 but is at least closer than air, i.e., n1<n3<n2. In such a case, the spherical wave deformation is at least reduced an the focus smearing range 630 is more less than it would be without the matching secondary medium 565.

In practice, it is essentially impossible to find an ideal perfect index matching medium 565 to general human tissue n2. One preferred embodiment for medium 565 would be to use fresh meat (possibly non-human). In other preferred embodiments, a less perfect approximation (for n3 medium matching to n2) would be to use a fluid preferably selected from: water, or saline fluid similar to human plasma, or blood. Yet other preferred embodiments for medium 565 would be to use gels containing high concentration of biological cellular material, thereby mimicking to closer approximation both the material composition and cellular subdivision structure of human tissue (each of which have manifest effects of varying dominance at different frequencies as highlighted in the “background” section discussion of FIG. 4).

As indicated in FIG. 4, we note that the average index of refraction n2 of the human body is in fact frequency dependent, i.e., n2=n2(w) varies with the frequency “w” of the radiation. Commonly all materials index of refraction varies with frequency. The method and apparatus of the present invention are optimized the closer the index matching medium n3 is closer in value to n2 at the particular frequency of electromagnetic radiation in which the device is operated.

For the case that the index matching medium 565 is selected to be water, the graph 565 shows that there is relatively good match between the refraction index n3 of water and the index n2 of a typical human body tissue only above 6.5 MHz. It is expected that the same is true also for saline, plasma, or blood fluids. Therefore, we conclude that in preferred embodiments of the present invention the electromagnetic radiation source is operated at frequencies above 6.5 MHz.

If particular applications of the present invention call for operation at frequencies lower than 6.5 MHz, then in preferred embodiments the appropriate index matching medium is selected to be such that its index of refraction n3 is matching to the n2 of human tissue at that selected frequency of operation. The putative approximate values of said index n3 can be inferred from the graph of FIG. 4 with the conversion n2˜[Re(ε)]^{1/2}.

FIG. 5 schematically illustrates the present invention system including integration of the index matching element with the elliptical reflecting surface 110. As illustrated, for clarity, we discuss the invention in terms of operation on a human brain region, but this is not meant to be limiting as any human body tissue can be similarly implemented. The invention method of inducing deep brain tissue stimulation (DBS) is based on:
(a) providing an elliptical reflecting surface/mirror 110 (or subsection 110 of an elliptical mirror) having one geometrical focal point F1 and a second geometrical focal point F2; providing a relatively small source 135 capable of emitting radiation of selected electromagnetic waves (e.g., an emission antenna); providing a secondary medium 565 confined under a spherical sub-section shell 560, said spherical shell being substantially transparent to the electromagnetic waves emitted by the source 135;
(b) placing said electromagnetic radiation source 135 at one of the geometric focal points (F1) 121 of said elliptical mirror 110 outside of the body of a human subject;
(c) placing said medium 565 confined under a spherical sub-section shell 560, such that the geometrical center of said shell 560 is coinciding with said focal point F2 of said elliptical reflecting surface 110;
(d) placing a pre-selected body organ region 190 for treatment (e.g., brain region, cancer tumor region, etc. . . . ) of the subject at position overlapping the second geometrical focal point (F2) 122 of said elliptical mirror 110;
(e) adjusting the volume content of the medium 565 such that its bottom surface 566 is tightly conforming to the contact surface of the body of said human subject;
(f) in operation, reflecting at least a portion of the emitted radiation from said elliptical mirror 110; thereby getting the emitted radiation from source 135 to be significantly concentrated at the region of said F2 focal point within said selected body organ region of said human subject.
(g) operating the radiation source 135 emission according to a pre-selected protocol for medical treatment of a pre-selected condition of said human subject; said protocol specifying at least the radiation frequency, intensity, and duration of said radiation.

As illustrated in FIG. 5(a), in order to hold the confining spherical section shell 560 positioned such that its geometrical sphere center coincides with the geometrical focal point F2 of the elliptical surface 110, there is some form of rigid connector element between them represented by rod 550 in FIG. 5(a). The rod shape of the rigid connector element 550 is only illustrative and is not meant to be limiting.

In position, the spherical shell side of index matching secondary medium 565 is facing towards the source 135 at focal point F1 of the reflecting elliptical surface 110 while the other side of medium 565 is facing towards the other focal point F2 which in operation is to be located within the human subject body. In order to minimize the distortion of the radiation wave front upon entering the human subject body, it is preferred that the secondary medium 565 contour will be as snugly fitting as possible to the contour of the subject body at the contact area. Therefore, as illustrated in FIG. 5(b), in preferred embodiments, the side of medium 565 which is facing towards F2 is bounded with a malleable or elastic boundary 566, and the contained medium 565 is itself malleable or elastic. Thereby it conforms to the human subject body contour when pressed against it.

In order for allowing variable positioning of the focal point F2 within a human subject body. Variable sizes of the spherical shell 560 and volume content of the medium 565 is possible. In preferred embodiments, the volume of the medium 565 contained between the shell 560 and bottom boundary 566 can be varied by inflow or outflow of the medium n3 volume via an inlet lumen 570.

In many instances we shall discuss the invention with respect to action targeted at brain tissue. Any such reference to brain stimulation, is not meant to be limiting, and should be regarded as a canonical example to application of the invention in any body tissue in general and nerve tissue in particular.

In particular embodiments, devices according to the invention can be used for focused deep brain stimulation (DBS) of selected brain regions with minimal effect on undesired brain regions. If placed outside the skull of a subject, is capable of stimulating the brain of the subject, including deep regions of the brain. Methods for using this device include treating human subjects with neurophysiological conditions, such as clinical or non-clinical depression, substance abuse, drug addiction, and other uses of DBS as known in the art of electromagnetic brain stimulation.

In order to preferentially stimulate selected deep brain regions, with minimum undesired effect on other brain regions, it key to have concentrated well peaked field. i.e., fields distribution where the field amplitude at the peak region is significantly bigger than away from the peak within the brain.

In addition, the invention relates to methods for inducing particular brain-wave states in selected brain regions.

Generation of said concentrated electric field in the brain can be used to generate local heating of brain regions. Optionally, said local electric fields, currents, and heating can be used to affect local blood clotting during hemorrhage or surgical wounds.

We recognize that the failure of prior art to produce well peaked deep brain electromagnetic fields stems from prior art non-invasive brain simulation reliance on producing an initial distribution of static magnetic fields. In contrast, we use focused electromagnetic waves (i.e., alternating fields) to induce local alternating currents in the brain.

All brain stimulation is actually generated by electric fields, not magnetic fields. In the prior art of so-called “magnetic brain stimulation” (MBS), it is the short duration transient electric fields (generated with a sharp turning off of the static magnetic field) which create the brain stimulation. In contrast, the present invention generates focused alternating electromagnetic fields of any duration and any desired intensity pattern.

As illustrated in FIG. 2, the invention method of inducing deep brain stimulation (DBS) is based on: introducing a relatively small source 135 emitting electromagnetic waves (i.e., an emission antenna) outside of a subject scull 190; and placing said source at one of the geometric focal points (F1) 121 of an elliptical mirror 110 (or subsection of an elliptical mirror), reflecting at least a portion of the emitted radiation from said elliptical mirror 110; placing the head 190 of an animal subject in a location such that a pre-selected brain region of the subject is located at the second geometrical focal point (F2) 122 of said elliptical mirror 110; thereby creating a focused image of the source 135 radiation at the region of said F2 focal point within the brain of said subject animal.

The mathematical absolute precision of elliptical shape and focal points should not be taken as limiting. More generally, according to the invention, a finite size electromagnetic source (e.g., antenna) placed overlapping one focal point of an elliptical mirror section will generate a maximum convergence of the emitted electromagnetic energy (i.e., peak of electromagnetic waves amplitude) in the neighborhood region of the other focal point of said ellipse. Thus said other focal point region is a convergence focus for the emitted electromagnetic radiation from the source placed at said first focus of the elliptical mirror. In practice, the noted characteristics of the above physical process remain also when the mirror shape deviates slightly from mathematically exact ellipse as is expected to occur in any manufacturing process of finite practical precision.

The theoretical physics basis of the method is the following: while static magnetic field are not reflected and not concentrated by a mirror, electromagnetic waves are reflected from a conducting mirror. Unlike common parabolic reflecting mirror which concentrate parallel light beams to a focus, elliptical mirrors create an image of radiation from one focal point to another focal point of the ellipse. Therefore, electromagnetic fields emitted from a source located at around one focal point (F1) of an elliptical mirror will generate a focused image of these electromagnetic waves at the other focal point (F2) of said elliptical mirror. Thus if a subject brain region is placed at the said F2 focal point then the alternating electric fields will induce local alternating electric fields of the same frequency within that brain region—thereby affecting it.

Local nerve stimulation in prior art was generally reliant on invasive electrode placement at the neighborhood of the target nerve, and influence the nerve activity by currents emanating from the electrode. Such nerve stimulation has found many medical applications. Since electromagnetic fields can induce local currents, in present invention we claim to provide non-invasive method of focusing of electromagnetic to influence nerve activity, and thereby provide non-invasive medical treatment methods to the same ends as with prior art invasive electrodes.

For example, the prior art of brain stimulation described in US patent application 2006/0200206 is comprising of an electrodes implanting procedure; involving positioning first and second electrodes at the identified stimulation site, and a stimulating procedure involving applying an electrical current between the first and second electrodes. Instead, the present invention can be implemented to the same treatment ends, by replacing the electrode stimulation with focused electromagnetic stimulation to convergence focal point F2 positioned to reside at the identified stimulation site.

Moreover, while electrode placement is fixed in place (and any electrode relocation is an added invasive procedure), the present invention convergence focus can be moved from place to place (and even smoothly along a continuous path) with no resistance and no need for repeated invasive procedures.

In preferred embodiments of the present invention for nerve stimulation, the radiation frequency is selected to be at an absorption resonance of said neuronal tissue, i.e., within a range of half-width of a local peak absorption frequency.

A condition for the electromagnetic waves to penetrate to deep brain regions is that the frequency or group of frequencies of the electromagnetic fields is selected to be such that is not significantly attenuated when traveling through the scull and brain tissue. Known examples of such frequency ranges are those of the well known EEG brainwaves bands between 1-40 Hz; and radio waves, from 3 MHz to 10 GHz (3 cm), are also known to be transmitted well though body tissue (e.g., including as in such uses as RFID typical devices). To be precise, the skin depth (δ) is defined as the penetration distance at which the field decreases to 1/e=0.368 of its value just inside the scull boundary surface. Approximating the human body as a “good conductor” we can use the equations

$$δ = \frac{1}{{ω [ \frac{μ e}{2} ( \sqrt{1 + {p}^{2}} - 1 ) ]}^{2 / 2}} , \\ p = \frac{σ}{ωε} >> 1 \\ and \\ δ = \frac{1}{\sqrt{nf μ α}} \qquad ( Eq . 1 )$$

The average human body conductivity is σ=0.5[S/m]. Thus at 1 Mhz the average δ˜1/[πx10⁶×4 π10⁻⁷×(0.5)]^{1/2}˜1 m. From this characteristic value, since the skin depth equation is proportional to [1/f]^{1/2}, we can approximate for 100 MHz a skin depth of δ˜10 cm, and for 10 GHz a skin depth of δ˜1 cm. In the published literature (see the book “Electromagnetic Shielding” By Kenneth L. Kaiser) we found that for human fat the range p=0.37 to 1.4 gives a 30 MHz skin depth of about 0.6 m to 2.2 m. Similarly for muscle tissue we found in the literature (Arumugam & Engels, “Characterization of RF Propagation in muscle Tissue for Passive UHF RFID Tags”, 2008) the value p=0.59. When p is in this range of values then a better approximation is to take not δ˜[1/f]^{1/2} but δ˜1/f. Hence, at frequency f=300 MHz we get another estimate skin depth of δ˜6 cm to 22 cm. Altogether, we reach the conclusion that for frequencies up to 500 MHz there is good penetration of electromagnetic waves to any location in the human body. Yet, also at around 500 MHz there begins to be significant absorption and therefore heating of body tissue. (for comparison, microwave ovens operate at around 2.5 GHz; cellphones commonly operate at 900 MHz to 1800 MHz bands).

We note that for focusing of plane waves by a lens there is a well known physical limit that maximum focusing of waves is limited a spot with a peak half height width “D” of size equal to about the wavelength λ of the wave. We note that in many of the above applications the preferred frequency is quite low, with respective wavelength bigger than the desired focus spot width D. An advantage of the invention method is that for spherical wave there is no physical limit to the focus peak width to be smaller than the radiation wavelength.

### BRIEF DESCRIPTION OF THE DRAWINGS

The invention is herein described, by way of example only, with reference to the accompanying drawings, wherein:

FIG. 1 illustrates the basic properties of elliptical mirrors.

FIG. 2 illustrates a system according to the present invention.

FIG. 3 illustrates changed positioning and movement of the focused deep brain stimulation location by system according to the present invention.

FIG. 4 illustrates a graph of the real part of the dielectric constant of both water (solid line) and typical human body tissue (dashed line).

FIG. 5 illustrates a preferred embodiment of the invention including an index matching secondary medium.

FIG. 6. Illustrated the physical principles of operation of the index matching secondary medium.

### DETAILED DESCRIPTION OF EMBODIMENTS

Before explaining at least one embodiment of the invention in detail, it is to be understood that the invention is not limited in its application to the details of construction and the arrangement of the components set forth in the following description or illustrated in the drawings.

Unless otherwise defined, all technical and scientific terms used herein have the same meaning as commonly understood by one of ordinary skill in the art to which this invention belongs. The materials, dimensions, methods, and examples provided herein are illustrative only and are not intended to be limiting.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations of these embodiments will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

Throughout, the term “stimulation” is meant to refer to any induced change of activity level, including but not limited to: increased activity, suppressed activity, and increased temperature. For example, “brain stimulation” is referred to both increased brain activity, inhibited brain activity, and combination thereof as measured by various parameters.

### Preferred Embodiments

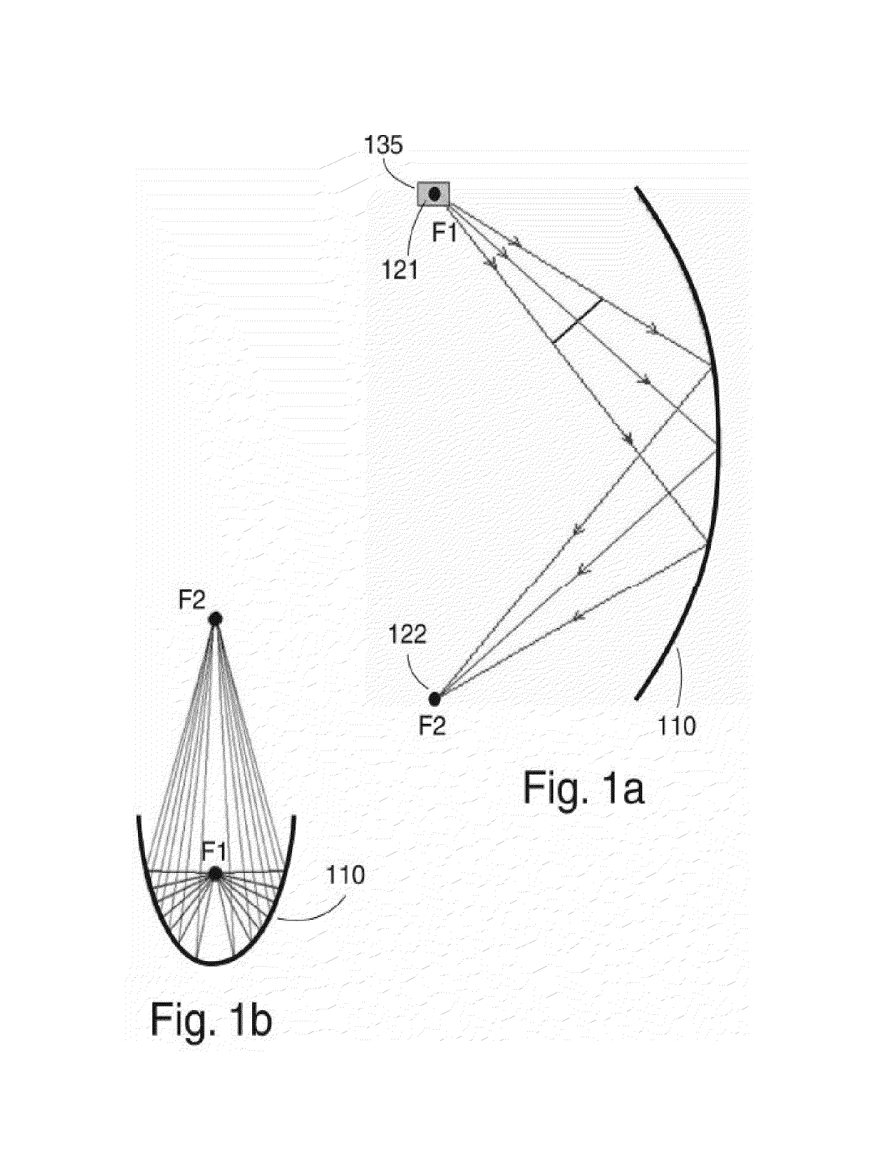

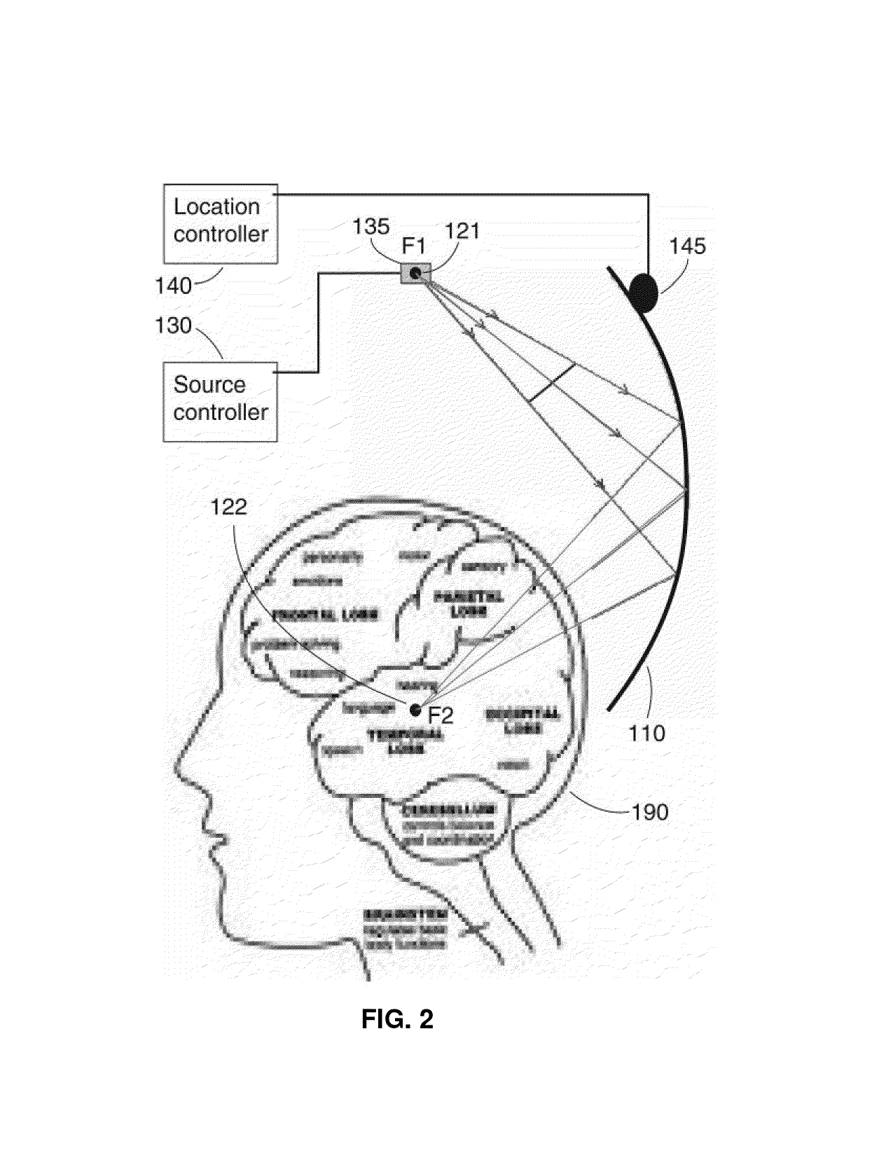

As illustrated in FIG. 1, the invention is based on the unique focusing properties of an elliptically curved reflecting surface; the radiation from a source 135 positioned at one focal point (F1) 121 of said elliptically curved surface is refocused at the second geometrical focal point (F2) 122 of said reflecting surface.

As illustrated in FIG. 1, it is understood that the elliptically curved surface need not be a full ellipse, but that the above noted property is true for any partial section of an ellipse. The incompleteness of a 3D elliptical mirror surface only implies that only some fraction of the emitted radiation energy from F1 will be refocused to F2, while some non reflected fraction not refocused to F2.

Any electromagnetic radiation source is applicable, e.g., any type of emitting antenna as known in the art (e.g., line, coil, etc. . . . ). In the context of the present invention, any multi-pole source should be placed such that its geometrical multipole center is located at the first focal point (F1) 121 of the elliptical mirror 110.

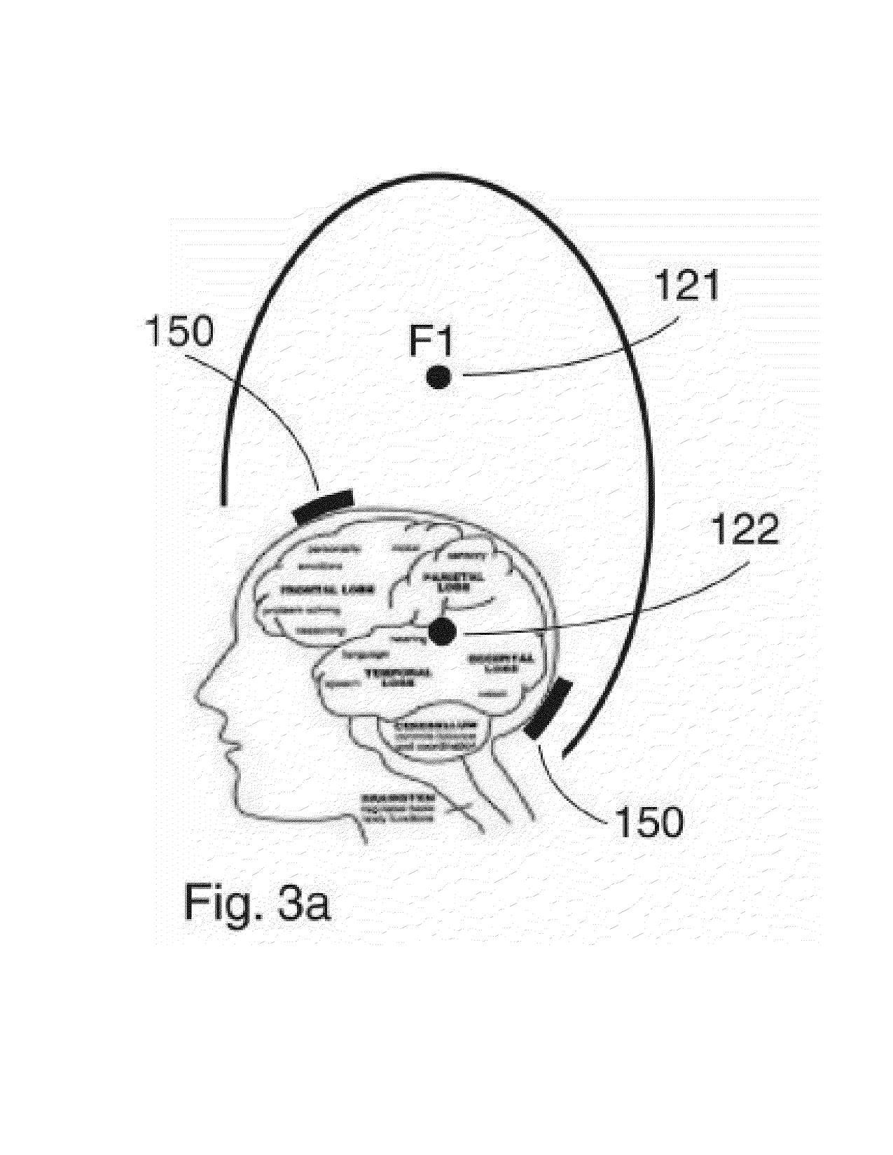

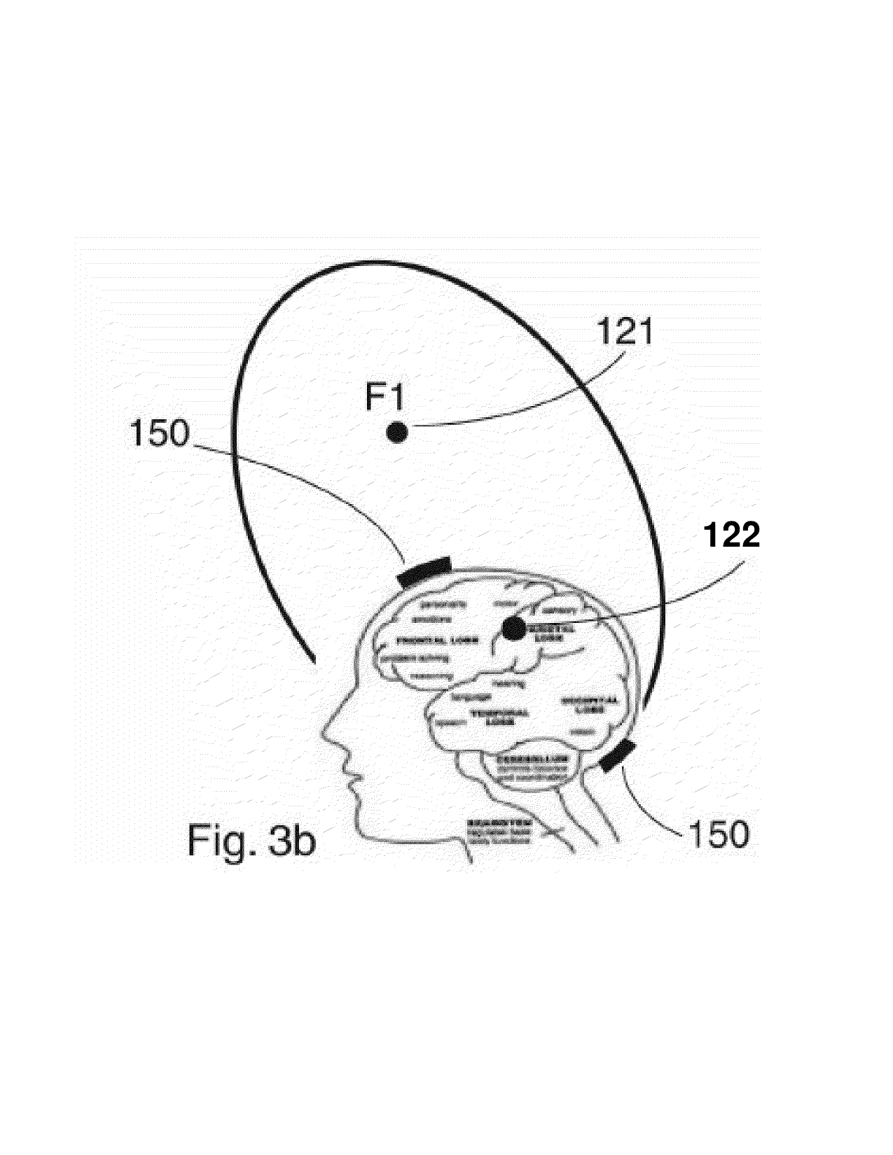

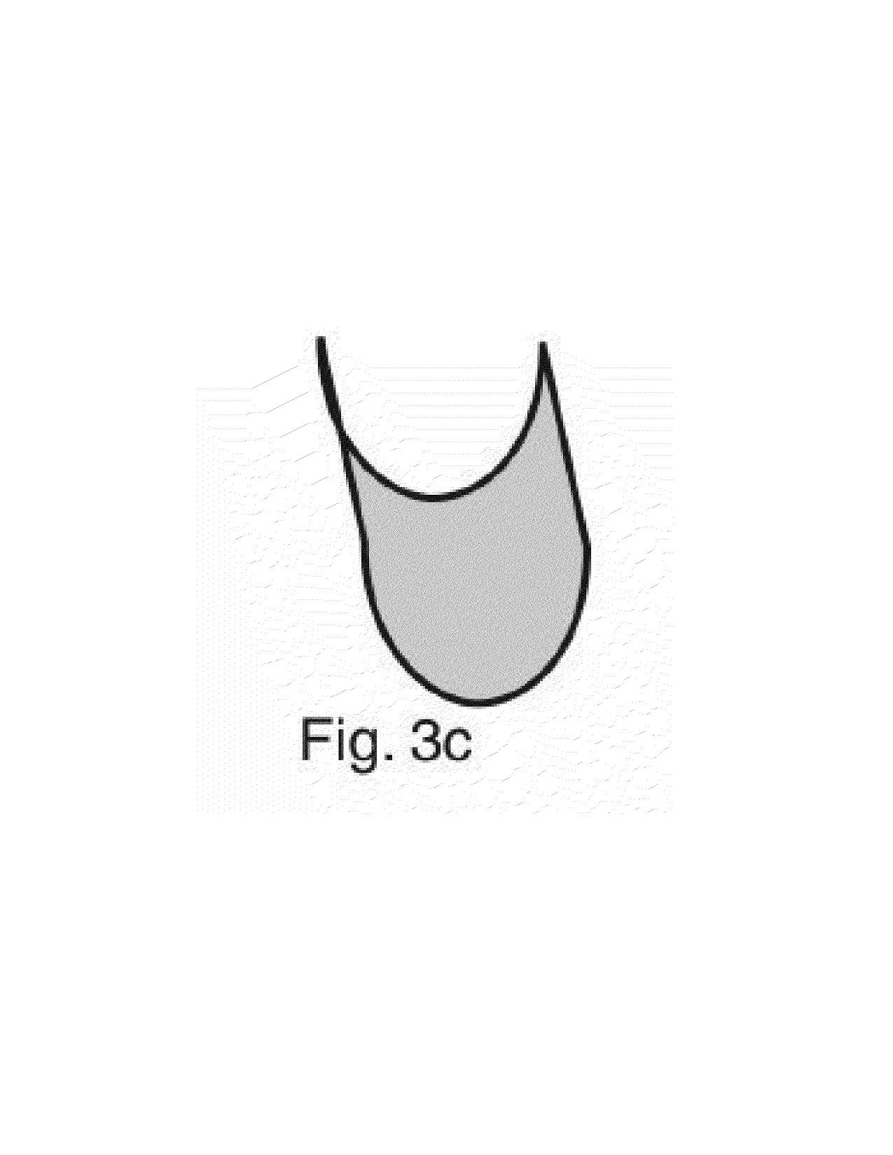

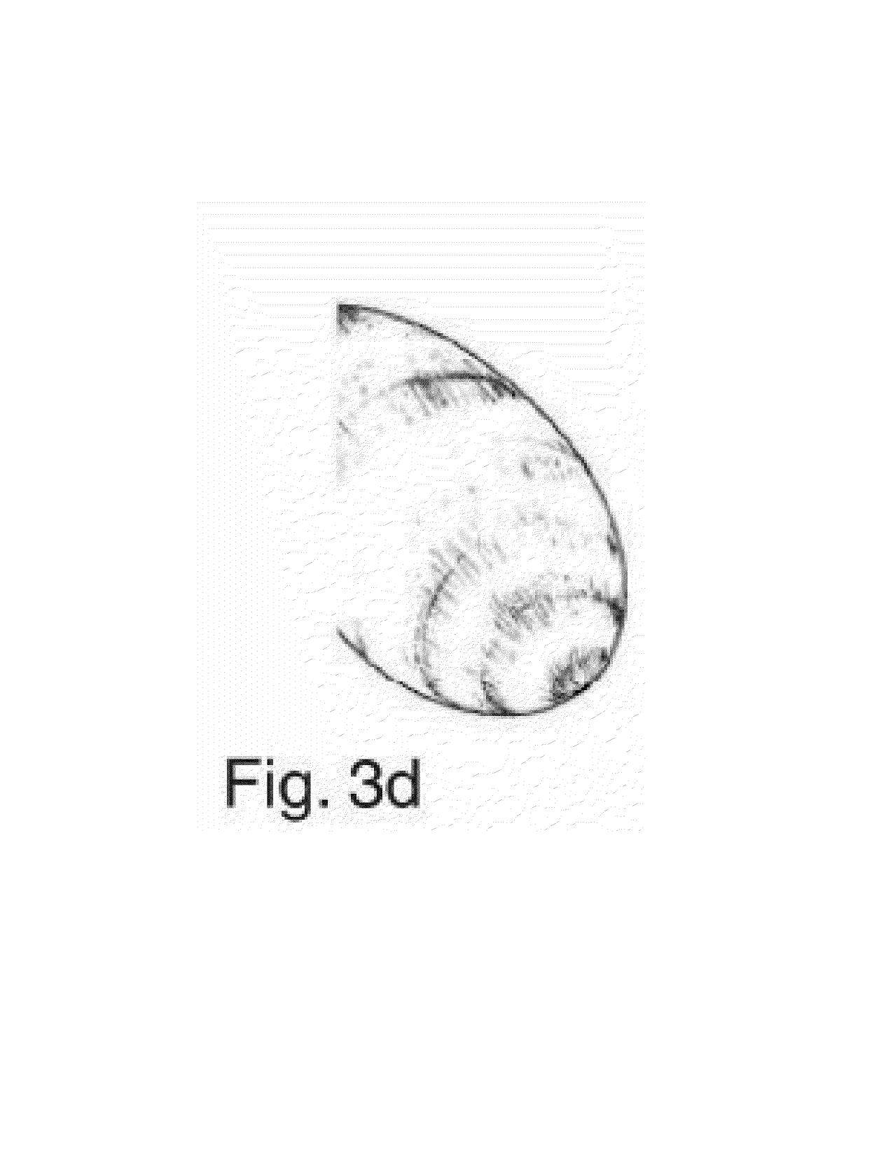

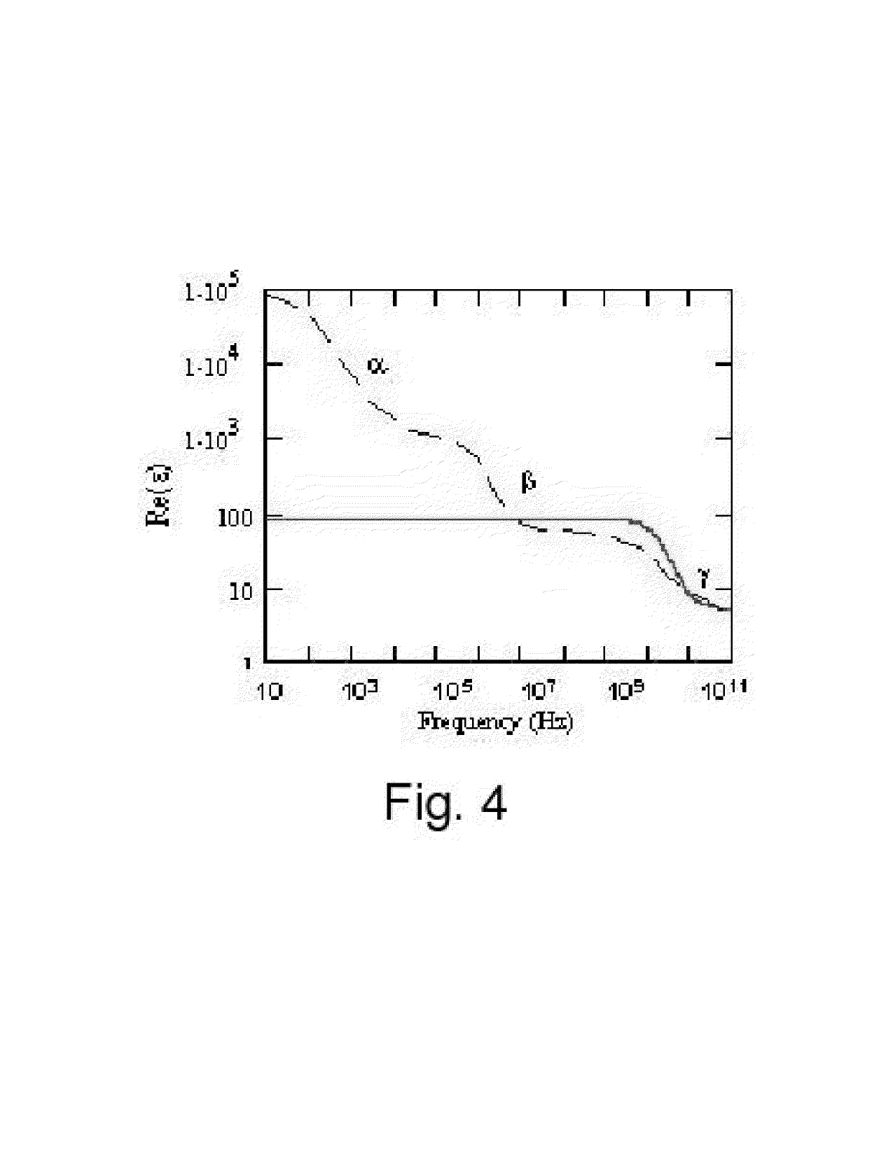

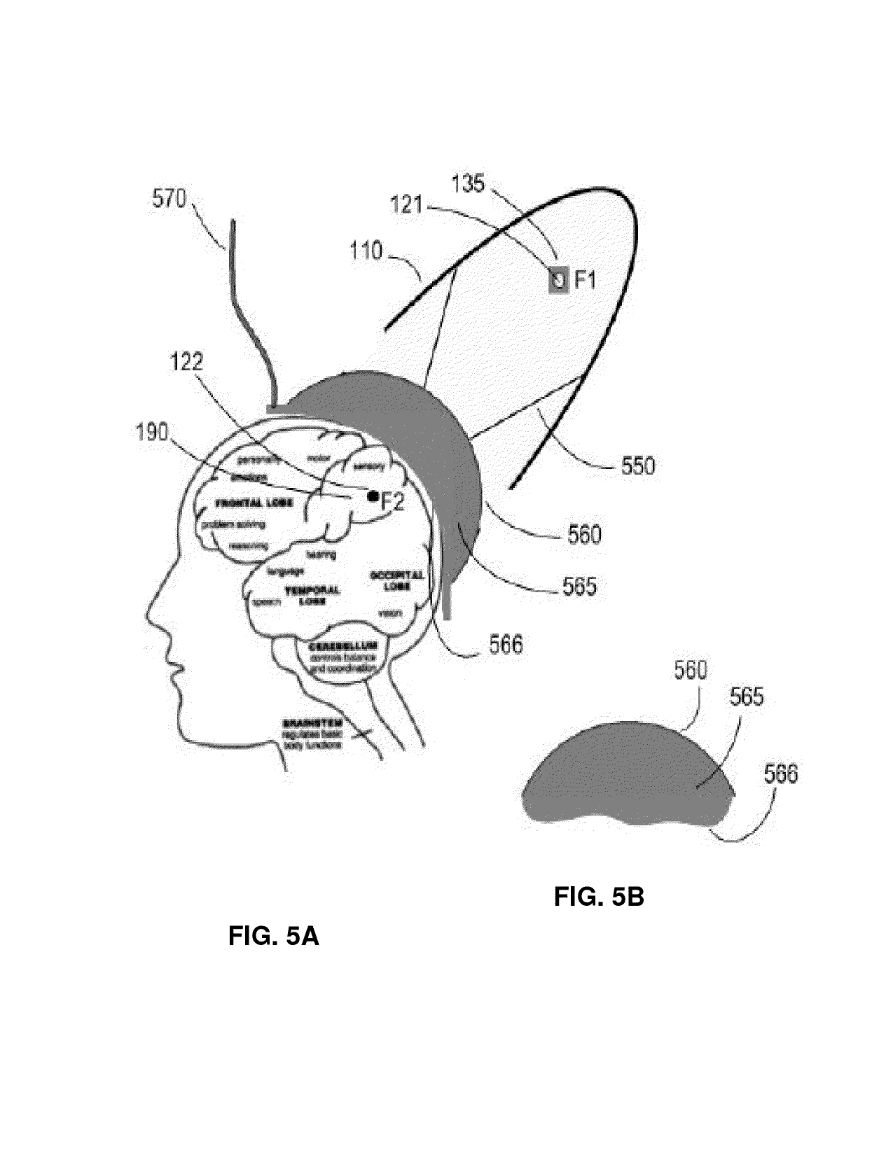

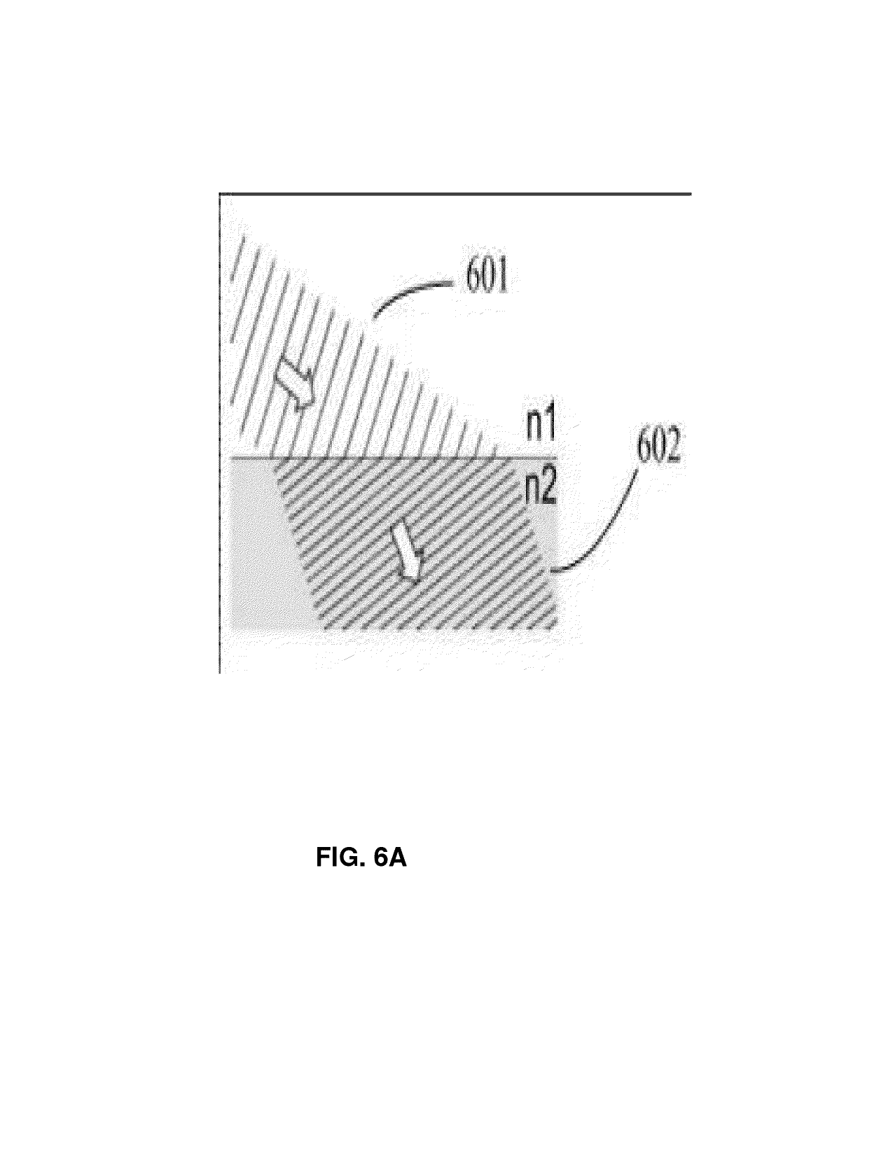

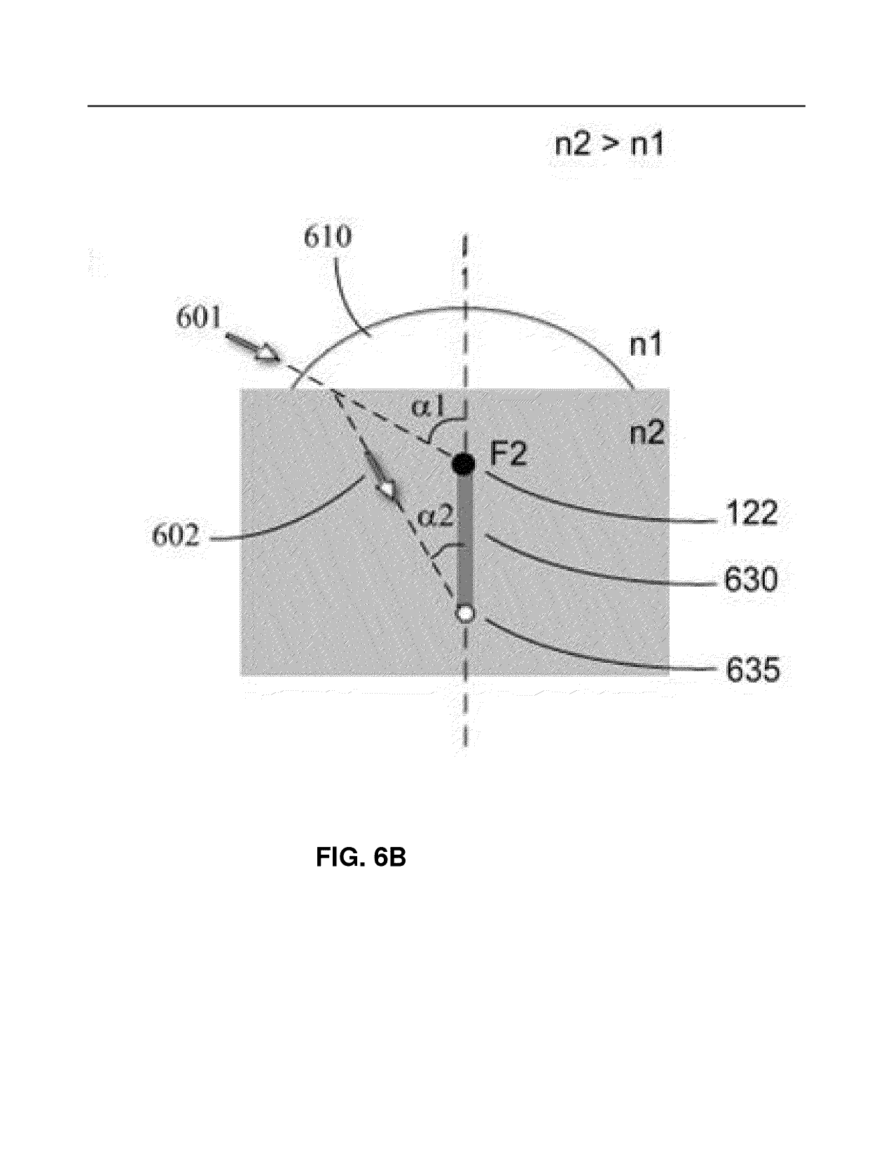

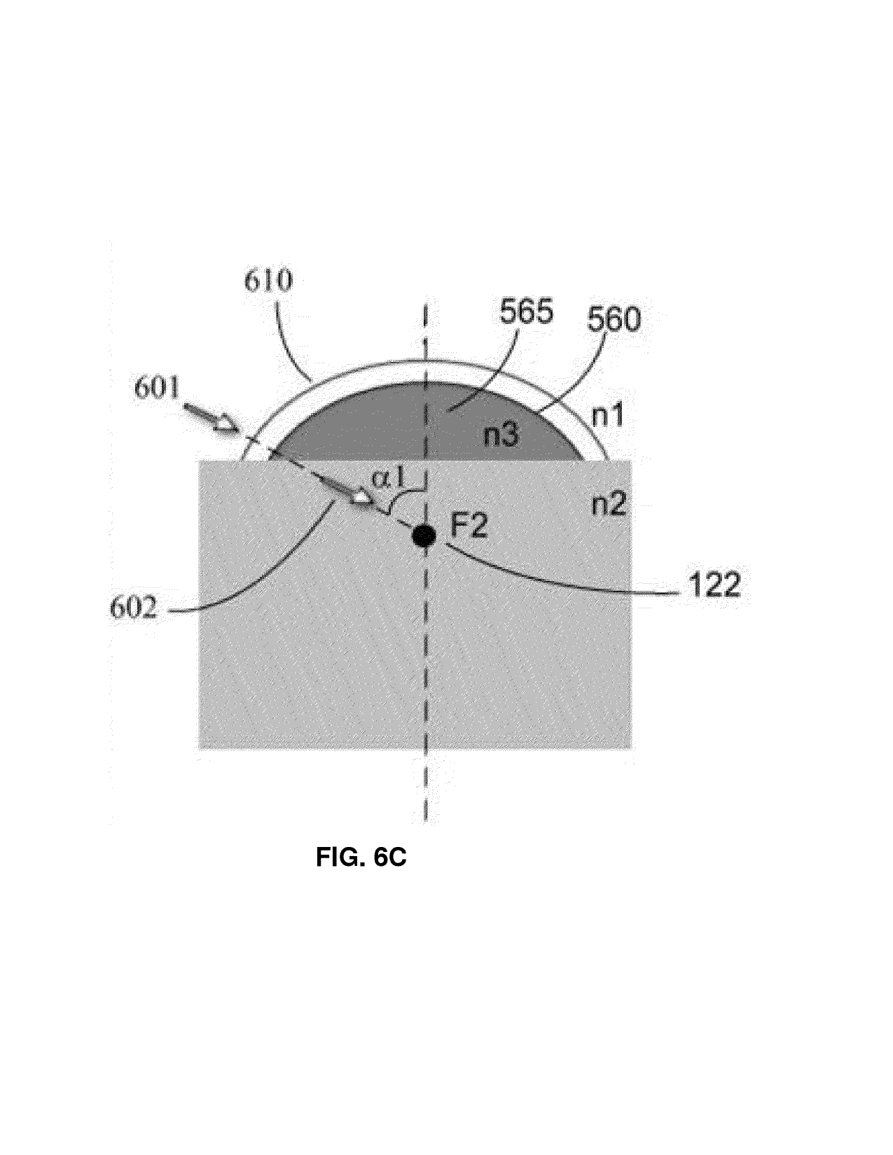

As illustrated in FIG. 3c and FIG. 3d, the elliptical mirrors as referred to in the present invention are 3D mirror structures. In preferred embodiments these elliptical mirrors may take various forms such as (a) an ellipsoid generated by rotating an ellipse about the axis connecting the two focal points, as illustrated in FIG. 3c, or (b) a “longitudinal ellipse” generated by parallel transporting an ellipse along a line path, as illustrated in FIG. 3d. For embodiments implementing the ellipsoid shape mirror it is preferred to use a small as possible “point like” sources in F1 focal point 121, such as a short coil (preferably of dimensions less than ⅕ the diameter of the ellipsoid at the plane of the focal point, the smaller the ratio the better radiation energy concentration is achieved the F2 convergence focus 122). For embodiments implementing the longitudinal ellipse shape mirror it is preferred to use an elongated “line like” source, such as a line antenna or a long coil, where such line source is placed along the geometrical line created by connecting the focal points of said parallel transported ellipse.

In order to reach flexibly to various deep tissue locations in a human subject, in preferred embodiments of the present invention, the distance between the focal points F1 and F2 of the reflecting surface 110 is larger than 5 cm.

In preferred embodiments of the present invention, the distance between the focal points F1 and F2 of the reflecting surface 110 is less than 50 cm.

A mathematical elliptical surface is characterized by the semi-major and semi-minor axis. In preferred embodiments of the present invention, both the semi-major and semi-minor axis are larger than 5 cm and smaller than 100 cm.

As illustrated in FIG. 3a,b, in preferred embodiments of the present invention, the body part of a human is held in place by some restricting elements (e.g., head holders 150) while the reflecting surface 110 is movable with respect to it in a controlled manner (e.g., by a motor element 145). Thereby, the convergence focal point F2 can be moved (either abruptly or smoothly) from one location to another in a human subject body.

In order to encircle large body parts such as the head, e.g., as illustrated in FIG. 3, both the semi-major and semi-minor axis are larger than 20 cm.

Maximum focusing to the convergence focal point F2 area is from emission locations as close as possible to the mathematically precise first focal point F1. Therefore, in preferred embodiments of the present invention, the emission source is selected to be small enough such that at least 90% of the emitted electromagnetic radiation from the source 135 is emitted from within 1 cm around the focal point F1 of the surface 110.

### Neural Stimulation—

Field strengths in preferred embodiments are selected such that they produce sufficient induced currents in brain to result in neuronal depolarization.

The electric field developed across the resting membrane of neural cells is around 10⁷ V/m. Hence, it may be required in preferred embodiments that the source fields at the first focal point will be of comparable magnitude. In preferred embodiments, such high fields may be generated by high field capacitors. For example, various ceramic based capacitors can withstand around 1 MV/cm (i.e., 10⁸ V/m) fields, and hence can be suited for use as high field electromagnetic radiation sources.

In preferred embodiments, the source 135 is a magnetic dipole source, such as a coil.

In preferred embodiments, neural stimulation is operated at frequencies corresponding to electromagnetic absorption resonances of the neural tissue. Some selected such resonance frequencies known in the published literature are: brain wave states in the range 4-15 Hz, and resonances in 10-50 Hz and 100-250 Hz frequency bands [Prog Brain Res. 2005; 148:181-8],

Highly localized neural stimulation by focused electromagnetic radiation of the present invention can be implementing as non-invasive method to the same application as electrode stimulation of neurons. For example, stimulation of brain blood flow by stimulating selected neurons or neural regions as elaborated in US patent application 20040220644 the content of which is here incorporated in its entirety.]

### Brainwaves State Enhancement—

The present invention also introduces a method and apparatus for enhancing particular brainwaves states in local areas of the brain. In use, the elliptical mirror 110 is placed such that the F2 convergence focus 122 is located within the desired target brain area for induction of brain waves. In preferred embodiments, the source is selected to emit one or more frequencies in the ranges of: delta waves lie in the frequency range of 0 to 3.5 Hz; theta waves lie in the frequency range of 4 to 7 Hz; alpha waves lie in the frequency range of 8 to 13 Hz; beta waves lie in the frequency range above 13 HHz; and sensorimotor rhythm (SMR) waves lie in the frequency range of 12 to 15 Hz.

### RF Ablation—

The present invention also introduces the first method of non-invasive radio-frequency ablation surgery. The term “radiofrequency (RF) ablation probe” refers to a class of medical devices operating between 460-550 kHz that deliver therapeutic energy into soft tissues. The intent of these devices is to thermally necrose tissue by raising targeted tissue temperatures to approximately 100° C. for a period of 10-15 minutes [1,2]. Instead of present art ablation probes being inserted percutaneously or subdermally (e.g., into tissues where cancerous tumors have been identified), the present invention creates a focused image at a focal point inside the body of a RF source placed outside the body at the other focal point of an elliptical mirror. The elliptical mirror serves to transmit the RF radiation energy from the source to the focal point inside the body. At 500 kHz, liver conductivity is approximately 0.148 S/m. By comparison, liver conductivity at 27 MHz and 2.45 GHz are 0.382 and 1.687 S/m.

### Local Hyperthermia—

The invention also introduces a non-invasive method and apparatus of inducing local hyperthermia of cancer tissue, by focusing of electromagnetic radiation. The intent of the electromagnetic radiation hyperthermia devices (preferably operating at 27 MHz or 2.45 GHz) is to raise the temperature of timorous tissues to between 43-45° C. for extended periods of time on the order of hours.

In use, the elliptical mirror 110 is placed such that the F2 convergence focus 122 is located within the desired target human organ for hyperthermia.

### Blood Coagulation Stimulation

The invention also introduces a non-invasive method and apparatus for inducing enhanced blood coagulation. In use, the elliptical mirror 110 is placed such that the F2 convergence focus 122 is located within the desired target human tissue for treatment, and where said location is anywhere under the skin. Such target tissue may comprise of surgical cuts, brain hemorrhage, and other internal body parts in need of prevention or inhibition of bleeding.

The radiation source power at F1 is selected so that the power transferred to the F2 convergence focus is such as not to raise the temperature of the surrounding tissues to such high values as to cause collapse of the tissue of the blood vessel. In other words an object is to obtain that the temperature transmitted by the electromagnetic radiation to the tissue to be coagulated never exceeds 70-75° C.

Also advantageously the resonance frequency of radiation is preferably but not necessarily chosen around 4 MHz. In preferred embodiments a combination of other frequencies is employed, e.g., a modulating wave may have the frequency, for instance 50 or 60 Hz or a frequency of 20-30 KHz.

The presence of a spectrum of harmonics in the resulting wave causes the manipulator to transmit a power and therefore an energy to the tissue under coagulation, which is the sum of the different specific energies due to the various frequencies. This is particularly important because at each molecule of the cellular tissue to be coagulated of different nature corresponds an ideal energy to be transmitted to reach in the present case, the correct temperature allowing transformation of the fibrinogen into fibrin without causing damages to the other adjacent cells.

What is claimed is:

1. A noninvasive method for providing focused tissue stimulation to a human patient, comprising of
performing a procedure to determine the location of target tissue at which a desired stimulation is to be performed;
positioning a source of electromagnetic radiation at first focal point of an elliptically curved reflecting surface, placing both said reflecting surface and said first focal point outside of said patient body;
positioning said target tissue to overlap with the second focal point of said elliptically curved reflecting surface;
applying an electrical current signal to said source to generate radiation of electromagnetic radiation at one or more selected frequencies;
continuing said radiation of a predetermined treatment session duration to affect said tissue;

2. A noninvasive method of inducing deep tissue stimulation, comprising of
(a) providing an elliptical reflecting surface/mirror 110 (or subsection 110 of an elliptical mirror) having one geometrical focal point F1 and a second geometrical focal point F2; providing a relatively small source 135 capable of emitting radiation of selected electromagnetic waves (e.g., an emission antenna); providing a secondary medium 565 confined under a spherical sub-section shell 560, said spherical shell being substantially transparent to the electromagnetic waves emitted by the source 135;
(b) placing said electromagnetic radiation source 135 at one of the geometric focal points (F1) 121 of said elliptical mirror 110 outside of the body of a human subject;
(c) placing said medium 565 confined under a spherical sub-section shell 560, such that the geometrical center of said shell 560 is coinciding with said focal point F2 of said elliptical reflecting surface 110;
(d) placing a pre-selected body organ region 190 for treatment (e.g., brain region, cancer tumor region, etc. . . . ) of the subject at position overlapping the second geometrical focal point (F2) 122 of said elliptical mirror 110;
(e) adjusting the volume content of the medium 565 such that its bottom surface 566 is tightly conforming to the contact surface of the body of said human subject;
